(19) United States
(12) Patent Application Publication
(10) Pub. No.: US 2009/0181366 A1
(43) Pub. Date: Jul. 16, 2009

(54) INTERNAL POSITIVE CONTROL FOR NUCLEIC ACID ASSAYS

(75) Inventors: Edgar B. Ong, San Diego, CA (US); Maurice Exner, Mission Viejo, CA (US); Michael A. Lewinski, San Clemente, CA (US)

Correspondence Address: FOLEY & LARDNER LLP, P.O. BOX 80278, SAN DIEGO, CA, 92138-0278, US

(73) Assignee: Quest Diagnostics Investments Incorporated

(21) Appl. No.: 11/830,759

(22) Filed: Jul. 30, 2007

(51) Int. Cl. C12Q 1/68

(52) U.S. Cl. 435/6

(57) ABSTRACT

Compositions and methods for detecting a non-specific nucleic acid amplification inhibitor in a reaction are disclosed. An internal positive control (IPC) may be included in samples to be tested for target nucleic acids as a means to monitor non-specific inhibition of nucleic acid amplification and provide confidence in negative results obtained in target-specific assays. Provided herein are an IPC polynucleotide, IPC control primers, and IPC probes. Also provided are methods of using an IPC polynucleotide, primers, and probes to detect a non-specific nucleic acid amplification inhibitor.

### FIELD OF THE INVENTION

The present invention relates generally to the fields of molecular diagnostics and nucleic acid amplification. In particular, the present invention relates to an internal positive control that may be used in nucleic acid amplification assays for detecting diseases or pathogens.

### BACKGROUND OF THE INVENTION

The following description is provided to assist the understanding of the reader. None of the information provided or references cited is admitted to be prior art to the present invention.

Various nucleic acid amplification techniques are known in the art, See e.g. Holland et al. (1991) PNAS 88: 7276, 7280; and U.S. Pat. No. 5,210,015. One assay uses a probe having a fluorescence reporter molecule and quencher molecule pair that is cleaved apart during amplification thereby resulting in a detectable fluorescent molecule in a concentration that is proportional to the amount of double-stranded DNA. These assays are known as TaqMan® based assays and permit the real-time detection of nucleic acid amplification. Furthermore, if multiple fluorophores are used, amplification from multiple nucleic acids can be detected simultaneously in the same reaction vessel. This allows for the detection of one or more targets, in addition to an internal positive control (IPC). See, e.g., U.S. Pat. No. 5,952,202.

Nucleic acid assays for the detection and identification of diseases and pathogens depend upon reliable and efficient amplification of the target molecules. It is important that measures are taken to ensure that false positives and negatives are distinguished from true positives and negatives. False negatives may occur due to technical error when non-specific inhibitors are present in the sample being tested. Inhibitors are frequently present in clinical samples and prevent the target nucleic acid from being amplified, even though it is present in the sample. As a result, the sample is identified as negative for the target, which may have serious consequences for the diagnosis and treatment of one or more diseases or conditions.

### SUMMARY OF THE INVENTION

The present inventors have discovered a nucleic acid template which may be used with complementary primers and probes as an IPC in nucleic acid amplification (e.g., PCR-based) assays. The IPC may be qualitative-erecting the presence or absence of a non-specific inhibitor- or quantitative-indicating the magnitude of the effect of the non-specific inhibitor on the amplification of one or more target sequences. Thus, the IPC of the present invention may be used to distinguish a true negative result from a false negative result, which incorrectly indicates that a sample lacks a target nucleic acid sequence. Furthermore, the IPC of the present invention may be used in molecular diagnostics since it does not exhibit homology with mammalian genes or known target nucleic acids used in disease or pathogen identification. Accordingly, there will be no amplification from endogenous sequences present in a mammalian sample.

In one aspect, the present invention relates to a control nucleic acid which can be used to measure the effect of a non-specific amplification inhibitor on the amplification of one or more target nucleic acids, thereby quantitating the amount of the target nucleic acids in a mammalian sample. Preferably, the control nucleic acid is not derived from mammals or known mammalian pathogens (e.g. a plant gene sequence), so that no amplification from an endogenous mammalian target or pathogen is detected. In such methods, the amount of a target nucleic acid present in a sample is quantified by (a) contacting a control nucleic acid with the mammalian sample to be assayed for the presence of a target nucleic acid, wherein the control nucleic acid comprises a nucleotide sequence from a plant gene; (b) amplifying the control nucleic acid with a control primer pair which is complementary to the control nucleic acid; (c) amplifying the target nucleic acid with one or more target primer pairs which are complementary to the one or more target nucleic acids; and (d) measuring the amount of an amplification product produced by the control primer pair and by the target primer pair. A reduction in the amount of the amplification product produced by the control primer pair relative to the amount of amplification product produced under similar conditions by the control primer pair without a nucleic acid amplification inhibitor present indicates the effect of any non-specific inhibitor in the mammalian sample. For instance, the degree to which the amplification of the control primer pair is reduced in the amplification of the sample compared to a standard where a non-specific inhibitor is known to be absent, is an indicator of the effect of the non-specific inhibitor on the amplification of the target nucleic acid. Thus, the amount of amplification product measured for the target primer pair may be adjusted to compensate for reduction in control nucleic acid amplified as a result of any non-specific inhibitor in the biological sample.

In one embodiment, the present invention relates to a control nucleic acid comprising from about 40 to about 1480 contiguous nucleotides of the gene encoding ribulose-1,5-bisphosphate carboxylase oxygenase large subunit N-methyltransferase (GenBank accession number BT005791, SEQ ID NO:1) of the plant Arabidopsis thaliana. In another embodiment, a 577-bp DNA fragment (SEQ ID NO: 2) derived from the SEQ ID NO: 1 is used as the IPC template.

In another aspect, the present invention provides a composition comprising an isolated control nucleic acid; wherein the control nucleic acid comprises from about 40 to about 1480 contiguous nucleotides of SEQ ID NO: 1 or its complement; a control primer pair which is complementary to the control nucleic acid; and, optionally, a labeled oligonucleotide probe complementary to the control nucleic acid. The control nucleic acid may comprise a fragment of SEQ ID NO: 1 having the nucleotide sequence according to SEQ ID NO: 2. The control primer pair may comprise any oligonucleotides complementary to the control nucleic acid capable of directing amplification of the control nucleic acid, or a detectable fragment thereof. For example, one or both of the primers of the control primer pair may be selected from the group consisting of SEQ ID NO: 5, SEQ ID NO: 6, SEQ ID NO: 8, and SEQ ID NO: 9 (See Table 1).

In some embodiments, the IPC composition comprises an oligonucleotide probe complementary to the control nucleic acid. Typically, the probe specifically hybridizes to a portion of the product amplified by the control primer pair and may comprise one or more labels (e.g. a fluorophore and/or a quencher) for the detection of the amplification product. For example, the oligonucleotide probe may comprise a sequence selected from the group consisting of: SEQ ID NO: 7 and SEQ ID NO:10 (See Table 1).

In certain embodiments, the IPC composition comprises an amount of the control nucleic acid that does not interfere with the simultaneous amplification of one or more target nucleic acids. In order to prevent the amplification reagents from becoming limiting during the reaction, the composition may comprise less than 1,000,000 copies, less than 750,000 copies, less than 600,000 copies, less than 500,000 copies, less than 400,000 copies, less than 300,000 copies, less than 100,000 copies, or less than 50,000 copies of the control nucleic acid. Typically, the composition will contain at least 100, at least 1,000, at least 5,000, or at least 10,000 copies of the control nucleic acid. In suitable embodiments, the composition comprises from about 300,000 to about 400,000 copies of the control nucleic acid.

In another aspect, the present invention provides qualitative methods of detecting a non-specific PCR inhibitor comprising: (a) contacting a control nucleic acid with a sample to be assayed for the presence of one or more target nucleic acids, wherein the control nucleic acid comprises from about 40 to about 1480 contiguous nucleotides of SEQ ID NO: 1; (b) amplifying the control nucleic acid by PCR with a control primer pair which is complementary to the control nucleic acid; and (c) detecting an amplification product produced by the control primer pair, wherein the presence of the amplification product produced by the control primer pair indicates that a non-specific PCR inhibitor is absent from the amplification mixture. The method may further comprise amplifying one or more target nucleic acids in the sample.

In some embodiments, the amplification is performed by PCR in real-time, wherein the amplification product is detected by measuring the presence or absence of a detectable label. The control primer(s) and/or an oligonucleotide probe may be labeled with a fluorophore, quencher, or other detectable moiety. The probe may be labeled at the 5′ end with a fluorescent dye and that hybridizes 3′ of the forward primer (i.e. a TaqMan® probe). The resulting amplicon is detected when the reporter dye is released by the action of the 5′-exonuclease activity of a DNA polymerase during amplification. The increase in fluorescence intensity during the reaction is detected and measured over time by a sequence detection instrument (e.g. Applied Biosystems ABI7000, ABI7500, ABI7700 and ABI7900HT). The primer and probe may also comprise the same molecule (i.e. a Scorpion™ primer/probe combination).

In some embodiments, the control nucleic acid is added to a sample to be tested for the presence of one or more target nucleic acids. During real-time PCR amplification, the control primer pair hybridizes within the IPC template to produce an amplicon. The amplicon is typically at least about 30, at least about 50, at least about 75, at least about 100, at least about 125, or at least about 150 nucleotides, and typically not more than about 500, not more than 400, or not more than about 300 nucleotides.

### DETAILED DESCRIPTION

Disclosed herein are compositions comprising a template, primers, and optionally, a probe, which may be used as an internal positive control (IPC) in nucleic acid assays. Also provided are methods for using the IPC in combination with diagnostic assays to detect one or more nucleic acids. The principal use of the IPC of the present invention is to detect the presence of a non-specific inhibitor in a sample during amplification of the relevant target sequence and to quantify the effect of that inhibitor on the amplification of one or more target nucleic acids. A true negative due to the absence of the target sequence must be distinguished from a false negative (inability to amplify a target sequence present in the sample due to the presence of inhibitors). A sample can be interpreted as a true negative only if the analysis of the IPC data indicates that nucleic acid amplification has occurred in the reaction tube. A negative PCR result for the IPC indicates the presence of an inhibitory substance in the sample being assayed that most likely contributed to the non-amplification of the target sequence. An IPC is an effective tool for monitoring inhibition of nucleic acid amplification and provides confidence in the negative results obtained with target-specific assays.

### Definitions

The present invention is described herein using several definitions, as set forth below and throughout the specification.

As used herein, unless otherwise stated, the singular forms “a,” “an,” and “the” include plural reference. Thus, for example, a reference to “an oligonucleotide” includes a plurality of oligonucleotide molecules, and a reference to “a nucleic acid” is a reference to one or more nucleic acids.

As used herein, “about” means plus or minus 10%.

The terms “amplification” or “amplify” as used herein includes methods for copying a target nucleic acid, thereby increasing the number of copies of a selected nucleic acid sequence. Amplification may be exponential or linear. A target nucleic acid may be either DNA or RNA. The sequences amplified in this manner form an “amplicon.” While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, Calif. 1990, pp 13-20; Wharam, et al., Nucleic Acids Res. 2001 Jun. 1; 29(11):E54-E54; Hafner, et al., Biotechniques 2001 April; 30(4):852-6, 858, 860; Zhong, et al., Biotechniques 2001 April; 30(4):852-6, 858, 860.

The term “complement,” “complementary,” or “complementarity” as used herein with reference to polynucleotides (i.e., a sequence of nucleotides such as an oligonucleotide or a target nucleic acid) refers to standard Watson/Crick pairing rules. The complement of a nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in natural nucleic acids may be included in the nucleic acids described herein; these include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be a sequence of RNA complementary to the DNA sequence or its complement sequence, and can also be a cDNA. The term “substantially complementary” as used herein means that two sequences specifically hybridize (defined below). The skilled artisan will understand that substantially complementary sequences need not hybridize along their entire length.

As used herein, “control nucleic acid” refers to a nucleic acid that is used as a template for amplification according to the methods disclosed herein. The term also includes nucleic acid fragments and may not necessarily comprise the full length sequence. In certain embodiments, a control nucleic acid fragment means any portion or amount less than the whole, including disjoined or non-contiguous portions. Typically, a fragment of a control nucleic acid sequence includes one or more contiguous sequences of at least about 10, at least about 40, at least about 50, at least about 75, at least about 100, at least about 150, or at least about 200 consecutive nucleotides, but not more than about 1500 consecutive nucleotides from the nucleic acid sequence. Such fragments may also include polymorphisms such that the fragment has at least 90% or at least 95% sequence identity to the same fragment of the nucleic acid without any polymorphisms.

As used herein, the term “detecting” used in context of detecting a signal from a detectable label to indicate the presence of a target nucleic acid in the sample does not require the method to provide 100% sensitivity and/or 100% specificity. As is well known, “sensitivity” is the probability that a test is positive, given that the person has a target nucleic acid sequence, while “specificity” is the probability that a test is negative, given that the person does not have the target nucleic acid sequence. A sensitivity of at least 50% is preferred, although sensitivities of at least 60%, at least 70%, at least 80%, at least 90% and at least 99% are clearly more preferred. A specificity of at least 50% is preferred, although sensitivities of at least 60%, at least 70%, at least 80%, at least 90% and at least 99% are clearly more preferred. Detecting also encompasses assays with false positives and false negatives. False negative rates may be 1%, 5%, 10%, 15%, 20% or even higher. False positive rates may be 1%, 5%, 10%, 15%, 20% or even higher.

The term “multiplex PCR” as used herein refers to simultaneous amplification of two or more products within the same reaction vessel. Each product is primed using a distinct primer pair. A multiplex reaction may further include specific probes for each product, that are detectably labeled with different detectable moieties.

The term “non-specific inhibitor” refers to an agent that inhibits or interferes with the amplification of nucleic acids in a way that does not depend on the sequence of the nucleic acid being amplified. Examples of such agents include, but are not limited to, hemoglobin, heparin, EDTA, humic acids, and fulvic acid. The term also refers to technical errors which interfere or prevent amplification from occurring in a reaction tube. Examples of technical errors include, but are not limited to, omission of an amplification reagent, unsuitable reaction/incubation conditions, or instrument failure.

As used herein, “nucleic acid,” “nucleotide sequence,” or “nucleic acid sequence” refer to a nucleotide, oligonucleotide, polynucleotide, or any fragment thereof and to naturally occurring or synthetic molecules. These phrases also refer to DNA or RNA of genomic or synthetic origin which may be single-stranded or double-stranded and may represent the sense or the antisense strand, or to any DNA-like or RNA-like material. An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose. RNA may be used in the methods described herein and/or may be converted to cDNA by reverse-transcription for use in the methods described herein.

As used herein, the term “oligonucleotide” refers to a short polymer composed of deoxyribonucleotides, ribonucleotides or any combination thereof. Oligonucleotides are generally between about 10, 11, 12, 13, 14 or 15 to about 150 nucleotides (nt) in length, preferably about 10, 11, 12, 13, 14, or 15 to about 70 nt, and more preferably between about 18 to about 30 nt in length. The single letter code for nucleotides is as described in the U.S. Patent Office Manual of Patent Examining Procedure, section 2422, table 1. In this regard, the nucleotide designation “R” means purine such as guanine or adenine, “Y” means pyrimidine such as cytosine or thymidine (uracil if RNA); and “M” means adenine or cytosine. An oligonucleotide may be used as a primer or as a probe.

As used herein, a “primer” for amplification is an oligonucleotide that is complementary to a target nucleotide sequence and leads to addition of nucleotides to the 3′ end of the primer in the presence of a DNA or RNA polymerase. The 3′ nucleotide of the primer should generally be identical to the target sequence at a corresponding nucleotide position for optimal expression and amplification. The term “primer” as used herein includes all forms of primers that may be synthesized including peptide nucleic acid primers, locked nucleic acid primers, phosphorothioate modified primers, labeled primers, and the like. As used herein, a “forward primer” is a primer that is complementary to the anti-sense strand of dsDNA. A “reverse primer” is complementary to the sense-strand of dsDNA.

Primers are typically between about 10 and about 100 nucleotides in length, preferably between about 12 and about 30 nucleotides in length, and most preferably between about 15 and about 25 nucleotides in length. There is no standard length for optimal hybridization or polymerase chain reaction amplification. An optimal length for a particular primer application may be readily determined in the manner described in H. Erlich, PCR Technology, Principles and Application for DNA Amplification, (1989).

An oligonucleotide (e.g., a probe or a primer) that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions.

“Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Equations for calculating Tm and conditions for nucleic acid hybridization are known in the art.

As used herein, an oligonucleotide is “specific” for a nucleic acid if the oligonucleotide has at least 50% sequence identity with a portion of the nucleic acid when the oligonucleotide and the nucleic acid are aligned. An oligonucleotide that is specific for a nucleic acid is one that, under the appropriate hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity. Sequence identity can be determined using a commercially available computer program with a default setting that employs algorithms well known in the art. As used herein, sequences that have “high sequence identity” have identical nucleotides at least at about 50% of aligned nucleotide positions, preferably at least at about 60% of aligned nucleotide positions, and more preferably at least at about 75% of aligned nucleotide positions.

Oligonucleotides used as primers or probes for specifically amplifying (i.e., amplifying a particular target nucleic acid sequence) or specifically detecting (i.e., detecting a particular target nucleic acid sequence) a target nucleic acid generally are capable of specifically hybridizing to the target nucleic acid.

As used herein, the term “sample” or “test sample” refers to any liquid or solid (or both) material that can be used to test for the presence of nucleic acids. Samples may comprise clinical samples, cells in culture or tissue cells, isolated nucleic acids, or isolated microorganisms. In preferred embodiments, a sample is obtained from a biological source (i.e., a “biological sample”), such as tissue, bodily fluid, or microorganisms collected from a subject. Sample sources include, but are not limited to, sputum (processed or unprocessed), bronchial alveolar lavage (BAL), bronchial wash (BW), blood, bodily fluids, cerebrospinal fluid (CSF), urine, plasma, serum, or tissue (e.g., biopsy material). The term “patient sample” as used herein refers to a sample obtained from a human seeking diagnosis and/or treatment of a disease.

As used herein, the term “Scorpion™ detection system” refers to a method for real-time PCR. This method utilizes a bi-functional molecule (referred to herein as a “Scorpion™”), which contains a PCR primer element covalently linked by a polymerase-blocking group to a probe element. Additionally, each Scorpion™ molecule contains a fluorophore that interacts with a quencher to reduce the background fluorescence.

The terms “target nucleic acid” or “target sequence” as used herein refer to a sequence which includes a segment of nucleotides of interest to be amplified and detected. Copies of the target sequence which are generated during the amplification reaction are referred to as amplification products, amplimers, or amplicons. Target nucleic acid may be composed of segments of a chromosome, a complete gene with or without intergenic sequence, segments or portions of a gene with or without intergenic sequence, or sequence of nucleic acids which probes or primers are designed. Target nucleic acids may include a wild-type sequence(s), a mutation, deletion or duplication, tandem repeat regions, a gene of interest, a region of a gene of interest or any upstream or downstream region thereof. Target nucleic acids may represent alternative sequences or alleles of a particular gene. Target nucleic acids may be derived from genomic DNA, cDNA, or RNA. As used herein target nucleic acid may be DNA or RNA extracted from a cell or a nucleic acid copied or amplified therefrom.

As used herein “TaqMan® PCR detection system” refers to a method for real time PCR. In this method, a TaqMan® probe which hybridizes to the nucleic acid segment amplified is included in the PCR reaction mix. The TaqMan® probe comprises a donor and a quencher fluorophore on either end of the probe and in close enough proximity to each other so that the fluorescence of the donor is taken up by the quencher. However, when the probe hybridizes to the amplified segment, the 5′-exonuclease activity of the Taq polymerase cleaves the probe thereby allowing the donor fluorophore to emit fluorescence which can be detected.

### IPC Nucleic Acids

Nucleic acids useful in the present invention exhibit certain features. One feature which is important for an IPC is that the IPC must be able to distinguish a true negative result from a false negative result. Thus, an IPC must be capable of robust amplification, but not so robust that it is amplified even in the presence of a non-specific inhibitor, when a target nucleic acid would not be amplified. Preferably, an IPC does not exhibit homology to any nucleic acid sequences which may be contained in a sample to be tested for one or more target nucleic acids. Consequently, when the sample to be tested is derived from a mammal, the IPC is not also derived from a mammal or any pathogens associated with mammals. Therefore, in some embodiments of the present invention, the IPC comprises a nucleic acid sequence from a plant gene, which does not exhibit any significant homology to a mammalian gene. A wide variety of gene sequences may be chosen, including, but not limited to genes involved in photosynthesis, flowering, enzymes required for cell wall biosynthesis, pathogen resistance R genes, and enzymes used for secondary metabolites in flowering plants. For example, a database of genes specifically expressed in the plant Arabidopsis thaliana, but not expressed in non-plant species, is described in Gutiérrez et al., Plant Physiology 135: 1888-1892 (2004). A skilled artisan is capable of choosing nucleic acid templates which possess the required feature using the description contained herein.

In some embodiments, the IPC template may be derived from gene encoding ribulose-1,5-bisphosphate carboxylase oxygenase large subunit N-methyltransferase (GenBank accession number BT005791, SEQ ID NO: 1) of the plant Arabidopsis thaliana. SEQ ID NO: 1 is as follows:

5′-ATGTCAGCCTCCGTCGCCGTCGTGTCGGGTTTTCTCCGTAT
CCCGTCAATTCAAAAGTCTCAAAACCCTTCCTTTCTCTTTTCTC
GACCCAAGAAATCTTTGGTGAGACCCATTTCAGCTTCAAGCTCA
GAGTTGCCGGAAAATGTTCGGAATTTCTGGAAATGGCTCAGAGA
CCAAGGAGTCGTATCCGGGAAGTCTGTGGCAGAACCTGCGGTGG
TACCGGAAGGTTTGGGACTTGTAGCCAGGCGTGATATTGGAAGA
AACGAGGTCGTATTGGAGATTCCCAAGCGATTGTGGATAAACCC
AGAGACAGTGACTGCTTCCAAGATTGGACCTTTATGCGGCGGAT
TAAAGCCGTGGGTTTCAGTAGCTCTGTTTTTGATCAGAGAGAAG
TATGAAGAAGAGTCTTCATGGAGAGTTTATCTTGATATGCTTCC
TCAATCTACTGATTCTACTGTCTTCTGGTCAGAAGAGGAGCTTG
CTGAGCTTAAAGGGACTCAACTGTTGAGCACCACATTGGGTGTG
AAAGAGTATGTGGAGAATGAATTCTTGAAACTGGAACAAGAGAT
ATTACTGCCTAACAAAGATCTCTTCTCATCCCGCATAACACTTG
ATGACTTCATATGGGCGTTTGGGATCCTCAAGTCGAGGGCTTTT
TCTCGTCTCCGTGGCCAAAACCTTGTCTTGATCCCTCTTGCAGA
CTTGATAAACCATAACCCCGCGATAAAGACAGAAGATTATGCAT
ACGAGATCAAAGGAGCCGGCCTTTTCTCTAGAGATCTCTTATTT
TCCTTGAAGTCACCTGTTTATGTTAAAGCAGGTGAGCAGGTATA
CATTCAGTACGATCTGAACAAAAGCAATGCAGAACTTGCTCTCG
ACTATGGTTTTGTGGAATCAAACCCTAAACGGAACTCATATACT
TTAACAATAGAGATACCAGAATCAGACCCATTCTTTGGGGATAA
GTTGGATATTGCTGAGAGTAACAAGATGGGTGAGACCGGATACT
TTGACATAGTAGACGGCCAGACTCTTCCCGCTGGTATGCTTCAG
TACCTTCGGCTTGTGGCTCTTGGCGGTCCAGATGCTTTCTTATT
AGAATCTATCTTCAATAACACCATATGGGGTCATCTTGAATTGC
CTGTAAGTCGTACAAACGAGGAACTCATATGCCGTGTTGTCAGA
GATGCCTGCAAATCTGCTCTGTCTGGTTTTGATACGACCATTGA
AGAGGATGAGAAGCTTCTGGACAAAGGAAAGCTTGAGCCTAGGT
TGGAAATGGCTCTCAAGATAAGGATTGGTGAGAAGAGAGTGCTT
CAGCAAATCGACCAAATCTTCAAGGATAGAGAGCTTGAACTTGA
CATTTTAGAGTATTACCAAGAGAGAAGGCTCAAAGATCTTGGGT
TGGTTGGCGAACAAGGGGATATTATCTTCTGGGAAACCAAGTGA
GAATTAGCTTAGCTTGTATAGCTTATCCTGC-3′

In an exemplary embodiment, the IPC template comprises SEQ ID NO: 2, which is a fragment of SEQ ID NO: 1. SEQ ID NO: 2 is as follows (shown in 5′→3′ orientation):

5′-TAACCCCGCGATAAAGACAGAAGATTATGCATACGAGATCA
AAGGAGCCGGCCTTTTCTCTAGAGATCTCTTATTTTCCTTGAAG
TCACCTGTTTATGTTAAAGCAGGTGAGCAGGTATACATTCAGTA
CGATCTGAACAAAAGCAATGCAGAACTTGCTCTCGACTATGGTT
TTGTGGAATCAAACCCTAAACGGAACTCATATACTTTAACAATA
GAGATACCAGAATCAGACCCATTCTTTGGGGATAAGTTGGATAT
TGCTGAGAGTAACAAGATGGGTGAGACCGGATACTTTGACATAG
TAGACGGCCAGACTCTTCCCGCTGGTATGCTTCAGTACCTTCGG
CTTGTGGCTCTTGGCGGTCCAGATGCTTTCTTATTAGAATCTAT
CTTCAATAACACCATATGGGGTCATCTTGAATTGCCTGTAAGTC
GTACAAACGAGGAACTCATATGCCGTGTTGTCAGAGATGCCTGC
AAATCTGCTCTGTCTGGTTTTGATACGACCATTGAAGAGGATGA
GAAGCTTCTGGACAAAGGAAAGCTTGAGCCTAGGTTGGAAATGG
CTCTCAAG-′3

### Amplification of Nucleic Acids

Nucleic acid samples or isolated nucleic acids may be amplified by various methods known to the skilled artisan. The nucleic acid (DNA or RNA) may be isolated from the sample according to any methods well known to those of skill in the art. If necessary the sample may be collected or concentrated by centrifugation and the like. The cells of the sample may be subjected to lysis, such as by treatments with enzymes, heat surfactants, ultrasonication or combination thereof. The lysis treatment is performed in order to obtain a sufficient amount of DNA from the sample to detect using polymerase chain reaction.

Various methods of DNA extraction are suitable for isolating the DNA. Suitable methods include phenol and chloroform extraction. See Maniatis et al., Molecular Cloning, A Laboratory Manual. 2d, Cold Spring Harbor Laboratory Press, page 16-54 (1989). Numerous commercial kits also yield suitable DNA including, but not limited to, QIAamp™ mini blood kit, Agencourt Genfind™, Roche Cobas® or phenol:chloroform extraction using Eppendorf Phase Lock Gels®.

Preferably, PCR is used to amplify nucleic acids of interest, i.e. one or more target nucleic acids and an IPC. Briefly, in PCR, two primer sequences are prepared that are complementary to regions on opposite complementary strands of the marker sequence. An excess of deoxynucleotide triphosphates are added to a reaction mixture along with a DNA polymerase, e.g., Taq polymerase.

For the IPC and the target sequence, if present in a sample, the primers will bind to the sequence and the polymerase will cause the primers to be extended along the target sequence by adding on nucleotides. By raising and lowering the temperature of the reaction mixture, the extended primers will dissociate from the marker to form reaction products, excess primers will bind to the marker and to the reaction products and the process is repeated, thereby generating amplification products. Cycling parameters can be varied, depending on the length of the amplification products to be extended.

In various embodiments of the present invention, oligonucleotide primers and probes are used in the methods described herein to amplify and detect target nucleic acids. A target nucleic acid can be a polymorphic region of a chromosomal nucleic acid, for example, a gene, or a region of a gene potentially having a mutation. Target nucleic acids include, but are not limited to, nucleotide sequence motifs or patterns specific to a particular disease and causative thereof, and to nucleotide sequences specific as a marker of a disease but not necessarily causative of the disease or condition. For example, target nucleic acids may include disease marker genes (including DNA and mRNA corresponding to the disease marker gene), single nucleotide polymorphisms, and microorganisms (i.e. bacteria and viruses). A target nucleic acid also can be a nucleotide sequence that is of interest for research purposes, but that may not have a direct connection to a disease or that may be associated with a disease or condition, although not yet proven so. The target nucleic acids may be detected simultaneously with an IPC added to the reaction, as described herein.

The skilled artisan is capable of designing and preparing primers that are appropriate for amplifying the IPC template in view of this disclosure. The length of the amplification primers for use in the present invention depends on several factors including the nucleotide sequence identity and the temperature at which these nucleic acids are hybridized or used during in vitro nucleic acid amplification. The considerations necessary to determine a preferred length for an amplification primer of a particular sequence identity are well known to the person of ordinary skill in the art.

Specifically, primers and probes to amplify and detect an IPC nucleic acid are provided by the invention. Primers that amplify a nucleic acid molecule can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection (e.g., by electrophoresis or real-time PCR), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 35 nucleotides in length, preferable 18-25 nucleotides in length. Exemplary primer sequences for use in the IPC of the present invention include SEQ ID NOS: 5, 6, 8, and 9.

Designing oligonucleotides to be used as hybridization probes can be performed in a manner similar to the design of primers. As with oligonucleotide primers, oligonucleotide probes usually have similar melting temperatures, and the length of each probe must be sufficient for sequence-specific hybridization to occur but not so long that fidelity is reduced during synthesis. Oligonucleotide probes are generally 15 to 60 nucleotides in length and may contain one or more detectable labels (i.e. fluorescent moieties and/or quenchers) to aid in erection. Such probes include, e.g. TaqMan® probes. Exemplary probe sequences for use in the IPC of the present invention include SEQ ID NOS: 7 and 10.

### Detection of Amplified Nucleic Acids

Amplification of nucleic acids can be detected by any of a number of methods well-known in the art such as gel electrophoresis, column chromatography, hybridization with a probe, sequencing, melting curve analysis, or “real-time” detection.

In one approach, sequences from two or more fragments of interest are amplified in the same reaction vessel (i.e. “multiplex PCR”). Detection can take place by measuring the end-point of the reaction or in “real time.” For real-time detection, primers and/or probes are detectably labeled with a fluorophore, for example, and amplified in an instrument capable of monitoring the change in fluorescence during the reaction. In end-point detection, the amplicon(s) could be detected by first size-separating the amplicons, then detecting the size-separated amplicons. The separation of amplicons of different sizes can be accomplished by, for example, gel electrophoresis, column chromatography, or capillary electrophoresis. These and other separation methods are well-known in the art. In one example, amplicons of about 10 to about 150 base pairs whose sizes differ by 10 or more base pairs can be separated, for example, on a 4% to 5% agarose gel (a 2% to 3% agarose gel for about 150 to about 300 base pair amplicons), or a 6% to 10% polyacrylamide gel. The separated nucleic acids can then be stained with a dye such as ethidium bromide and the size of the resulting stained band or bands can be compared to a standard DNA ladder.

In some embodiments, amplified nucleic acids are detected by hybridization with a specific probe. Probe oligonucleotides, complementary to a portion of the amplified target sequence may be used to detect amplified fragments. Hybridization may be detected in real time or in non-real time. Amplified nucleic acids for each of the target sequences may be detected simultaneously (i.e., in the same reaction vessel) or individually (i.e., in separate reaction vessels). In preferred embodiments, the amplified DNA is detected simultaneously, using two or more distinguishably-labeled, gene-specific oligonucleotide probes, which hybridize to one or more target sequences and one which hybridizes to the IPC sequence.

The probe may be detectably labeled by methods known in the art. Useful labels include, e.g., fluorescent dyes (e.g., Cy5®, Cy3®, FITC, rhodamine, lanthamide phosphors, Texas red, FAM, JOE, Cal Fluor Red 610®, Quasar 670®), ³²P, ³⁵S, ³H, ¹⁴C, ¹²⁵I, ¹³¹I, electron-dense reagents (e.g., gold), enzymes, e.g., as commonly used in an ELISA (e.g., horseradish peroxidase, beta-galactosidase, luciferase, alkaline phosphatase), colorimetric labels (e.g., colloidal gold), magnetic labels (e.g., Dynabeads™), biotin, dioxigenin, or haptens and proteins for which antisera or monoclonal antibodies are available. Other labels include ligands or oligonucleotides capable of forming a complex with the corresponding receptor or oligonucleotide complement, respectively. The label can be directly incorporated into the nucleic acid to be detected, or it can be attached to a probe (e.g., an oligonucleotide) or antibody that hybridizes or binds to the nucleic acid to be detected.

One general method for real time PCR uses fluorescent probes such as the TaqMan® probes, molecular beacons, and Scorpions. Real-time PCR quantitates the initial amount of the template with more specificity, sensitivity and reproducibility, than other forms of quantitative PCR, which detect the amount of final amplified product. Real-time PCR does not detect the size of the amplicon. The probes employed in Scorpion™ and TaqMan® technologies are based on the principle of fluorescence quenching and involve a donor fluorophore and a quenching moiety.

In a preferred embodiment, the detectable label is a fluorophore. The term “fluorophore” as used herein refers to a molecule that absorbs light at a particular wavelength (excitation frequency) and subsequently emits light of a longer wavelength (emission frequency). The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Förster or fluorescence resonance energy transfer (“FRET”). Quenching by FRET is generally used in TaqMan®, probes while proximal quenching is used in molecular beacon and Scorpion™ type probes.

In proximal quenching (a.k.a. “contact” or “collisional” quenching), the donor is in close proximity to the quencher moiety such that energy of the donor is transferred to the quencher, which dissipates the energy as heat as opposed to a fluorescence emission. In FRET quenching, the donor fluorophore transfers its energy to a quencher which releases the energy as fluorescence at a higher wavelength. Proximal quenching requires very close positioning of the donor and quencher moiety, while FRET quenching, also distance related, occurs over a greater distance (generally 1-10 nm). Thus, when FRET quenching is involved, the quenching moiety is an acceptor fluorophore that has an excitation frequency spectrum that overlaps with the donor emission frequency spectrum. When quenching by FRET is employed, the assay may detect an increase in donor fluorophore fluorescence resulting from increased distance between the donor and the quencher (acceptor fluorophore) or a decrease in acceptor fluorophore emission resulting from decreased distance between the donor and the quencher (acceptor fluorophore).

Suitable fluorescent moieties include the following fluorophores known in the art: 4-acetamido-4′-isothiocyanatostilbene-2,2′disulfonic acid, acridine and derivatives (acridine, acridine isothiocyanate) Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes), 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Black Hole Quencher™ (BHQ™) dyes (biosearch Technologies), BODIPY® R-6G, BOPIPY® 530/550, BODIPY® FL, Brilliant Yellow, coumarin and derivatives (coumarin, 7-amino-4-methylcoumarin (AMC, Coumarin 120), 7-amino-4-trifluoromethylcouluarin (Coumarin 151)), Cy2®, Cy3®, Cy3.5®, Cy5®, Cy5.5®, cyanosine, 4′,6-diamindino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin, diethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansyl chloride), 4-(4′-dimethylaminophenylazo)benzoic acid (DABCYL), 4-dimethylaminophenylazophenyl-4′-isothiocyanate (DABITC), Eclipse™ (Epoch Biosciences Inc.), eosin and derivatives (eosin, eosin isothiocyanate), erythrosin and derivatives (erythrosin B, erythrosin isothiocyanate), ethidium, fluorescein and derivatives (5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET)), fluorescamine, IR144, IR1446, Malachite Green isothiocyanate, 4-methylumbelliferone ortho cresolphthalein, nitrotytosine, pararosaniline, Phenol Red, B-phycoerythrin, R-phycoerythrin, o-phthaldialdehyde, Oregon Green®, propidium iodide, pyrene and derivatives (pyrene, pyrene butyrate, succinimidyl 1-pyrene butyrate), QSY® 7, QSY® 9, QSY® 21, QSY® 35 (Molecular Probes), Reactive Red 4 (Cibacron® Brilliant Red 3B-A), rhodamine and derivatives (6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, sulforhodamine B, sulforhodamine 101, sulfonyl chloride derivative of sulforhodamine 101 (Texas Red)), N,N,N′,N′-tetramethyl-6-carboxyrhodamine (TAMRA), tetramethyl rhodamine, tetramethyl rhodamine isothiocyanate (TRITC), riboflavin, rosolic acid, terbium chelate derivatives.

The detectable label can be incorporated into, associated with or conjugated to a nucleic acid. Label can be attached by spacer arms of various lengths to reduce potential steric hindrance or impact on other useful or desired properties. See, e.g., Mansfield, 9 Mol. Cell. Probes 145-156 (1995). Detectable labels can be incorporated into nucleic acids by covalent or non-covalent means, e.g., by transcription, such as by random-primer labeling using Klenow polymerase, or nick translation, or amplification, or equivalent as is known in the art. For example, a nucleotide base is conjugated to a detectable moiety, such as a fluorescent dye, and then incorporated into nucleic acids during nucleic acid synthesis or amplification.

TaqMan® probes (Heid, et al., Genome Res 6: 986-994, 1996) use the fluorogenic 5′ exonuclease activity of Taq polymerase to measure the amount of target sequences in cDNA samples. TaqMan® probes are oligonucleotides that contain a donor fluorophore usually at or near the 5′ base, and a quenching moiety typically at or near the 3′ base. The quencher moiety may be a dye such as TAMRA or may be a non-fluorescent molecule such as 4-(4-dimethylaminophenylazo) benzoic acid (DABCYL). See Tyagi, et al., 16 Nature Biotechnology 49-53 (1998). When irradiated, the excited fluorescent donor transfers energy to the nearby quenching moiety by FRET rather than fluorescing. Thus, the close proximity of the donor and quencher prevents emission of donor fluorescence while the probe is intact.

TaqMan® probes are designed to anneal to an internal region of a PCR product. When the polymerase (e.g., reverse transcriptase) replicates a template on which a TaqMan® probe is bound, its 5′ exonuclease activity cleaves the probe. This ends the activity of the quencher (no FRET) and the donor fluorophore starts to emit fluorescence which increases in each cycle proportional to the rate of probe cleavage. Accumulation of PCR product is detected by monitoring the increase in fluorescence of the reporter dye (note that primers are not labeled). If the quencher is an acceptor fluorophore, then accumulation of PCR product can be detected by monitoring the decrease in fluorescence of the acceptor fluorophore.

With Scorpion™ probes, sequence-specific priming and PCR product detection is achieved using a single molecule. Scorpion™ probes comprise a 3′ primer with a 5′ extended probe tail comprising a hairpin structure which possesses a fluorophore/quencher pair. The probe tail is “protected” from replication in the 5′ to 3′ direction by the inclusion of hexethlyene glycol (HEG) which blocks the polymerase from replicating the probe. The fluorophore is attached to the 5′ end and is quenched by a moiety coupled to the 3′ end. After extension of the Scorpion™ primer, the specific probe sequence is able to bind to its complement within the extended amplicon thus opening up the hairpin loop. This prevents the fluorescence from being quenched and a signal is observed. A specific target is amplified by the reverse primer and the primer portion of the Scorpion™, resulting in an extension product. A fluorescent signal is generated due to the separation of the fluorophore from the quencher resulting from the binding of the probe element of the Scorpion™ to the extension product. Such probes are described in Whitcombe et al., Nature Biotech 17: 804-807 (1999).

In a suitable embodiment, real time PCR is performed using any suitable instrument capable of detecting fluorescence from one or more fluorescent labels. For example, real time detection on the instrument (e.g. a ABI Prism 7900HT Sequence Detector) monitors fluorescence and calculates the measure of reporter signal, or Rn value, during each PCR cycle. The threshold cycle, or Ct value, is the cycle at which fluorescence intersects the threshold value. The threshold value is determined by the sequence detection system software or manually.

In some embodiments, melting curve analysis may be used to detect an amplification product. Melting curve analysis involves determining the melting temperature of a nucleic acid amplicon by exposing the amplicon to a temperature gradient and observing a detectable signal from a fluorophore. Melting curve analysis is based on the fact that a nucleic acid sequence melts at a characteristic temperature called the melting temperature (Tm), which is defined as the temperature at which half of the DNA duplexes have separated into single strands. The melting temperature of a DNA depends primarily upon its nucleotide composition. Thus, DNA molecules rich in G and C nucleotides have a higher Tm than those having an abundance of A and T nucleotides.

Where a fluorescent dye is used to determine the melting temperature of a nucleic acid in the method, the fluorescent dye may emit a signal that can be distinguished from a signal emitted by any other of the different fluorescent dyes that are used to label the oligonucleotides. In some embodiments, the fluorescent dye for determining the melting temperature of a nucleic acid may be excited by different wavelength energy than any other of the different fluorescent dyes that are used to label the oligonucleotides. In some embodiments, the second fluorescent dye for determining the melting temperature of the detected nucleic acid is an intercalating agent. Suitable intercalating agents may include, but are not limited to SYBR™ Green 1 dye, SYBR dyes, Pico Green, SYTO dyes, SYTOX dyes, ethidium bromide, ethidium homodimer-1, ethidium homodimer-2, ethidium derivatives, acridine, acridine orange, acridine derivatives, ethidium-acridine heterodimer, ethidium monoazide, propidium iodide, cyanine monomers, 7-aminoactinomycin D, YOYO-1, TOTO-1, YOYO-3, TOTO-3, POPO-1, BOBO-1, POPO-3, BOBO-3, LOLO-1, JOJO-1, cyanine dimers, YO-PRO-1, TO-PRO-1, YO-PRO-3, TO-PRO-3, TO-PRO-5, PO-PRO-1, BO-PRO-1, PO-PRO-3, BO-PRO-3, LO-PRO-1, JO-PRO-1, and mixture thereof. In suitable embodiments, the selected intercalating agent is SYBR™ Green 1 dye.

By detecting the temperature at which the fluorescence signal is lost, the melting temperature can be determined. In the disclosed methods, each of the amplified target nucleic acids may have different melting temperatures. For example, each of these amplified nucleic acids (target(s) and IPC) may have a melting temperature that differs by at least about 1° C., more preferably by at least about 2° C., or even more preferably by at least about 4° C. from the melting temperature of any of the other amplified nucleic acids. Detecting melting temperatures of the target amplicons and/or the IPC can confirm the presence or absence of the target in the sample.

To minimize the potential for cross contamination, reagent and mastermix preparation, specimen processing and PCR setup, and amplification and detection are all carried out in physically separated areas. Uracil-N-Glycosylase (UNG) may be utilized (along with the incorporation of uracil into PCR amplicons) to eliminate carry over contamination.

In a clinical or research setting, it may be desirable to accurately quantitate the amount of target nucleic acid in a sample. For example, to measure the viral or bacterial load in a sample or the level of gene expression (i.e. from a disease marker gene), accurate quantitation of the target nucleic acid is necessary. A non-specific inhibitor may entirely block amplification of a target nucleic acid, or it may reduce the efficiency of amplification, which would indicate that the sample contains less target than it actually does. Accordingly, in some embodiments, the present invention provides an IPC which is added in a known amount to a sample to be assayed for one or more target nucleic acids. The amplification of the IPC in the sample can be compared to a standard reaction where a non-specific inhibitor is known to be absent. If the amplification of the IPC in the sample is similar to that observed in the standard, then no non-specific inhibitor is present in the sample. Conversely, if the amplification of the IPC in the sample is less than that observed in the standard, then a non-specific inhibitor is present. Therefore, in quantitating the target nucleic acid in the sample, the amount observed in the amplification should be adjusted upward to the same extent as the reduction observed in the IPC amplification. For example, in a real-time PCR reaction, if the Ct of the IPC is n cycles greater in the sample compared to the standard, then the Ct of the target should be increased n cycles to compensate for the effects of the non-specific inhibitor.

### EXAMPLES

The present invention is further illustrated by the following examples, which should not be construed as limiting in any way.

### Example 1

### QIPC Primers and Probes

Table 1 shows sequences and design of the Quantitative Internal Positive Control (QIPC) primers and probes utilized in the Examples. All primers and probes were synthesized by Eurogentec North America (San Diego, Calif.), purified by HPLC, analyzed by mass spectrometry and redissolved in 1×TE buffer, pH 8 to a final concentration of 1 mM. The working concentration for the primers and probe is usually 100 μM. The stock and diluted primers were kept at −70° C. until required.

TABLE 1
QIPC Primers and Probe Sequences

| | | | |
|---|---|---|---|
| Name | Description | SEQ ID NO: | Sequence (5′→3′) |

| | | | |
|---|---|---|---|
| RUBL_01 | Cloning primer 1 | SEQ ID NO:3 | TAACCCCGCGATAAAGACAG |
| RUBR_01 | Cloning Primer 2 | SEQ ID NO:4 | CTTGAGAGCCATTTCCAACC |
| QIPC_L | QIPC1 PCR Primer 1 | SEQ ID NO:5 | CTTCAGTACCTTCGGCTTG |
| QIPC_R | QIPC1 PCR Primer 2 | SEQ ID NO:6 | TTGCAGGCATCTCTGACAAC |
| QIPC_XY | Probe 1 (QIPC1) | SEQ ID NO:7 | Reporter Dye-TGGCTCTTGGCG |
| | | | GTCCAGATG-Quencher Dye |
| QIPC2_L | QIPC2 PCR Primer 1 | SEQ ID NO:8 | CAGACTCTTCCCGCTGGT |
| QIPC2_R | QIPC2 PCR Primer 2 | SEQ ID NO:9 | ATCTGGACCGCCAAGAGC |
| QIPC2_XY | Probe 2 (QIPC2) | SEQ ID NO:10 | Reporter Dye-TGCTTCAGTACC |
| | | | TTCGGCTTGTG-Quencher Dye |
| QIPC2_VIC/NFQ | Probe 2′ (QIPC) | SEQ ID NO:11 | Reporter Dye (VIC)- |
| | | | TGCTTCAGTACC |
| | | | TTCGGCTTGTG-Quencher Dye |
| | | | (MGB/NFQ) |

The following dyes were used in this study: JOE or YAKIMA YELLOW (a substitute for the VIC™ dye) for 5′ reporter dye, and TAMRA or a non-fluorescent quencher (e.g. Eclipse Dark Quencher) for 3′ quencher dye. As an example, a probe with JOE at the 5′ end and TAMRA at the 3′ end will be called “QIPC_JT,” and a probe with YAKIMA YELLOW at the 5′ end and Eclipse Dark Quencher at the 3′ end will be called “QIPC_YE.” The absorption and emission wavelengths for the 5′ dyes are 520 and 548 nm, and 526 and 548 nm for JOE and YAKIMA YELLOW, respectively. TAMRA has an absorption wavelength of 560-565 nm and an emission wavelength of 580-582 nm. The Eclipse Dark Quencher has an absorption of 522 nm and has no emission wavelength as this quencher does not emit light.

### Example 2

### Cloning of IPC DNA from an Arabidopsis cDNA Library

This example describes the cloning and purification of the IPC DNA from an Arabidopsis plant cDNA library (obtained from Biochain Institute, Cat # A510289). The following components were combined in an amplification mixture:

TABLE 2
PCR Reaction Components

| | Stock | | | Final | |
|---|---|---|---|---|---|
| Component | Concentration | | Volume (μL) | Concentration | |
| Water | | | 37.75 | | |
| PCR Gold Buffer | 10x | | 5.00 | 1x | |
| dNTPs | 10 | mM | 1.00 | 0.2 | mM |
| RUBL_01 | 10 | μM | 2.50 | 0.5 | μM |
| RUBR_01 | 10 | μM | 2.50 | 0.5 | μM |
| Arabidopsis cDNA | 2.5 | ng/μL | 1.00 | 2.5 | ng |
| AmpliTaq Gold ® | 5 | U/μL | 0.25 | 1.25 | U |

PCR was performed using an MJ PCR instrument with the following cycling parameters: 95° C., 10 min; 40 cycles of 94° C., 30 sec; 53° C., 30 sec; 68° C., 1 min; and a final extension 68° C., 10 min. This reaction generated a 577-bp fragment (corresponding to SEQ ID NO: 2) which was purified using a QIAQuick® PCR purification kit (Qiagen).

### Example 3

### Preparation of Stock IPC DNA and Primer/Probe Mix

To produce sufficient amounts of QIPC DNA (SEQ ID NO: 2) for use in subsequent applications, the purified PCR product was reamplified using the same primer set as in Example 2 above on a 96-well PCR plate. After amplification and gel electrophoresis of the samples from 10 randomly chosen wells, samples from each well were combined and purified using QiaQuick PCR Purification Kit®. The final QIPC DNA solution was concentrated 10-fold using spin filter concentrators (Microcon-100 by Millipore). The amount of QIPC DNA was calculated using PicoGreen dsDNA Quantification Kit (Molecular Probes catalog no. P-7589) and a fluorescent reader. The final yield was 57.3 μg (equal to 9.2×10¹³ copies). The QIPC DNA solution was diluted further to create a 50× stock of 3.6×10⁸ copies/mL. A 10× mix of 100 μM (each) of the primers and probes was prepared. All solutions were stored at −70° C.

### Example 4

### PCR Efficiency of IPC

To determine the PCR efficiency of the IPC primers and probes, the PCR efficiency between parallel runs on the ABI7000 and ABI7900HT instruments, and the PCR efficiency of target primers (the AVIUM primers and probe for detecting Mycobacterium avium DNA) was tested in the presence of the QIPC primers, probe, and DNA. Different dilutions of the QIPC DNA were made in 1×TE buffer, pH 8. The primer and probe concentrations were set as described below. Each concentration for the QIPC DNA or target sequence (M. avium DNA) was tested with at least five replicates.

Samples with varying concentrations of QIPC DNA were tested on ABI7000, ABI7500 and ABI7900HT. Table 3 indicates that PCR efficiency of the QIPC primers (at 250 nM each) and QIPC_YE probe (at 50 nM) was at least 95% in ABI7000. Table 4 indicates that PCR efficiency did not change when the same primer/probe concentrations were tested on the ABI7900HT (it actually improved compared to that obtained on the ABI7000). Table 5 shows that PCR efficiencies of QIPC primers (at 54 nM each) and QIPC_YE, QIPC_JT or QIPC_JE (at 150 nM) using different combination of reporter and quencher dyes did not change and all had efficiencies greater than 95%.

Table 6 shows that PCR efficiency of the AVIUM primers (at 500 nM each) and probe (at 100 nM) was not affected by the presence of the QIPC DNA (50,000 copies/reaction), and QIPC primers (at 90 nM each) and QIPC probe (QIPC_YE at 20 nM). The data indicate that the primers have a 97% efficiency. Additionally the average Ct value for the QIPC did not change in the presence of very high copy number of target sequence, in this case 1E+7 copies of M. avium DNA. All negative (no template) samples were not detected.

TABLE 3
PCR Efficiency of QIPC Primers and Probe QIPC_YE

| | |
|---|---|
| QIPC DNA | QIPC |

| (copies/mL) | n | Avg Ct | % Positive |
|---|---|---|---|
| 1,000,000 | 5 | 26.398 | 100 |
| 100,000 | 5 | 29.960 | 100 |
| 10,000 | 5 | 33.234 | 100 |
| 1,000 | 5 | 27.258 | 100 |
| 100, | 5 | 37.723 | 100 |
| 10 | 5 | 41.138 | 80 |
| 0 | 5 | UD | 0 |

Linear Regression Line: y = −3.5854x + 47.847
R square: 09983
Efficiency: 1.901
% Efficiency: 95.034

TABLE 4
PCR Efficiency of QIPC Primers Between
ABI 7000 and ABI 7900HT instruments

| QIPC DNA | | ABI7000 | | ABI7900HT | |
|---|---|---|---|---|---|
| (copies/mL) | n | Avg Ct | % Positive | Avg Ct | % Positive |
| 1,000,000 | 5 | 26.398 | 100 | 27.617 | 100 |
| 100,000 | 5 | 29.960 | 100 | 30.524 | 100 |
| 10,000 | 5 | 33.234 | 100 | 33.490 | 100 |
| 1,000 | 5 | 27.258 | 100 | 37.204 | 100 |
| 100, | 5 | 37.723 | 100 | N/A | N/A |
| 10 | 5 | 41.138 | 80 | N/A | N/A |
| 0 | 5 | UD | 0 | UD | 0 |

| | | |
|---|---|---|
| Linear | y = −3.5854x + | y = −3.2585x + |
| Regression Line: | 47.847 | 46.801 |
| R square: | 09983 | 0.9978 |
| Efficiency: | 1.901 | 2.030 |
| % Efficiency: | 95.034 | 101.360 |

TABLE 5
PCR Efficiency of Different Probe Dye Combinations.

| QIPC DNA | | | |
|---|---|---|---|
| (copies/ml) | QIPC_YE | QIPC_JT | QIPC_JE |
| 0 | UD | UD | UD |
| 2,000 | 34.29 | 35.26 | 3453 |
| 10,000 | 31.98 | 32.83 | 32.35 |
| 20,000 | 30.90 | 31.54 | 31.15 |
| 60,000 | 29.15 | 30.01 | 29.45 |
| 100,000 | 28.45 | 29.18 | 28.67 |
| 160,000 | 27.92 | 28.48 | 28.06 |
| 200,000 | 27.62 | 28.13 | 27.63 |
| Linear | y = −3.3785x + | y = −3.5646x + | y = −3.4752x + |
| regression line | 45.433 | 47.013 | 46.093 |
| R square | 0.9987 | 0.9993 | 0.9989 |
| Efficiency | 1.977 | 1.908 | 1.940 |
| % Efficiency | 98.846 | 95.391 | 96.989 |

UD = Undetermined no amplification)

TABLE 6
PCR Efficiency of AVIUM Primers with QIPC (50,000 copies/rxn)

| M. avium DNA | | QIPC | | AVIUM | |
|---|---|---|---|---|---|
| (copies/ml) | n | Avg Ct | % Pos | Avg Ct | % Pos |
| 10,000,000 | 5 | 26.286 | 100 | 23.060 | 100 |
| 1,000,000 | 5 | 26.162 | 100 | 26.440 | 100 |
| 100,000 | 5 | 26.182 | 100 | 29.630 | 100 |
| 10,000 | 5 | 26.108 | 100 | 33.520 | 100 |
| 0 | 5 | 26.228 | 100 | UD | 0 |

UD = Undetermined (no amplification)
Linear regression line: y = −3.457x + 36.805
R square 0.9983
Efficiency 1.947
% Efficiency 97.328

### Example 5

### Optimization Study of QIPC Primer and Probe Concentration

To determine the optimum primer probe concentration still capable of generating accurate and reproducible results, different concentrations of primers and probe were prepared: 9, 18, 36, 54, 72, 90 nM primers, and 10, 50, 150, and 250 nM probe (QPC-JT), Studies indicated that different combinations of reporter (JOE or YAKIMA YELLOW) and quencher dyes (TAMRA or Eclipse Dark Quencher) in the QIPC probe worked equally well (Table 5). Three replicates for each primer/probe concentration set were tested. The QIPC DNA was set at 10,000 copies per reaction. All runs were performed using the ABI7500 instrument.

Three replicates for each primer/probe set were tested. Table 7 shows that a QIPC primer concentration greater than or equal to 36 nM provided an average Ct value. The probe concentration (between 10 to 250 nM) was not as critical and the Ct values ranged from 25.75 to 27.84 at 36 nM of primer concentration. Based on these results, the primer concentration for further experiments was initially set at 36 nM for each primer and the probe concentration was set at 150 nM.

TABLE 7
Optimization of QIPC Primer/Probe Concentration.

| Primer | | Probe concentration | | | |
|---|---|---|---|---|---|
| concentration | n | 250 nM | 150 nM | 50 nM | 10 nM |
| 90 nM | 3 | 24.873 | 24.787 | 25.673 | 27.617 |
| 72 nM | 3 | 24.910 | 24.937 | 25.533 | 27.737 |
| 54 nM | 3 | 24.187 | 24.760 | 25.253 | 27.180 |
| 36 nM | 3 | 25.833 | 25.750 | 26.390 | 27.843 |
| 18 nM | 3 | 34.200 | 29.490 | 29.953 | 31.033 |
| 9 nM | 3 | 36.297 | 37.913 | 37.717 | 38.500 |

### Example 6

### Optimization Study of QIPC DNA Concentration

To determine the optimum concentration of QIPC DNA still capable of generating accurate and reproducible results, different concentrations of QIPC DNA were prepared in 1×TE buffer, pH 8: 3.6E+8, 2.7 E+8, 1.8E+8, 9.0E+7, 3.6E+7, 3.6E+6, 3.6E+5, and 3.6E+4 copies/mL. The QIPC primer and probe (QIPC_JT) concentration used was 36 nM and 150 nM, respectively All runs were performed on the ABI7500 instrument.

Five replicates for each dilution were tested. Table 8 shows that the QIPC DNA concentration of 3.6E+8 copies/mL (equals 360,000 copies/reaction) gave an average Ct value of 27.500. For the rest of the validation study, the QIPC DNA concentration was set at 360,000 copies reaction.

TABLE 8
Optimization of QIPC DNA Concentration.

| QIPC DNA | | |
|---|---|---|
| (copies/mL) | n | Avg. Ct. |
| 3.6E+8 | 5 | 27.500 |
| 2.7E+8 | 5 | 30.888 |
| 1.8E+8 | 5 | 31.499 |
| 9.0E+7 | 5 | 32.524 |
| 3.6E+7 | 5 | 34.745 |
| 3.6E+6 | 5 | 38.150 |
| 3.6E+5 | 5 | UD |
| 3.6E+4 | 5 | UD |
| 0 | 5 | UD |

UD = Undetermined (no amplification)

### Example 7

### Analytical Study of Cross-Reactivity

To determine the specificity of the QIPC primers to hybridize uniquely to the QIPC DNA, QIPC primers (at 36 nM) and probe (QIPC_YE at 150 nM) were tested in the presence of M. avium DNA, M. intracellulare DNA, M. tuberculosis DNA, West Nile Virus RNA and nucleic acids from other pathogens (see text below Table 9). No nucleic acids from other targets were detected using QIPC primers (Table 9). Thus, the primers were specific for the QIPC DNA. Furthermore, GenBank sequence homology searches using BLAST indicate that there was no homology or significant homology with sequences from other organisms. Therefore, there was no cross-reactivity with the nucleic acids from these other organisms.

TABLE 9
Summary of Specificity Study

| Nucleic Acid Tested | n | Avg. Ct. |
|---|---|---|
| QIPC DNA | 3 | 25.958 |
| M. avium DNA | 3 | UD |
| M. intracellulare DNA | 3 | UD |
| M. tuberculosis DNA | 3 | UD |
| West Nile Virus RNA | 3 | UD |
| Pathogen Panel SS1* | 3 | UD |
| Pathogen Panel SS2** | 3 | UD |
| Water | 3 | UD |

UD = Undetermined (no amplification)
*Includes: B. pertussis, M. pneumoniae, varicella zoster virus, Epstein-Barr virus, C. pneumoniae, T. gondii, M. tuberculosis, human immunodeficiency virus 1, Legionella sp., L. pneumoniae, cytomegalovirus
**Includes: B. microti, human coronavirus, enteric coronavirus, rat coronavirus, influenza A virus, influenza B virus, Dengue fever virus, human herpes simplex virus 2

### Example 8

### Heparin Interference Study

It is known that heparin interferes with the polymerase chain reaction (Beutler et al., 1990, Biotechniques 9: 166). This study addressed the effect of various concentrations of heparin on the amplification of both target sequence and QIPC DNA in comparison with the an internal positive control product sold by ABI (“ABI IPC”) (catalog no. 4308323).

A green-top BD Vacutainer Sodium Heparin Plastic (catalog no. 367874) containing 150 USP units of sodium heparin was dissolved in 2.5 mL of elution buffer used in the MagNA Pure Total Nucleic Acid Isolation Kit. This concentration at 60 USP units/mL was set as 1×. Further dilutions of the heparin were made to obtain 0.5×, 0.2×, 0.1×, 0.01×, 0.001×, 0.0001×, and 0.00001×. The PCR reactions were performed as before except that 5 μl of the heparin solution was added to each reaction in a total volume of 50 μl. In this study 1E+5 and 1E+3 copies/mL of West Nile Virus RNA were used as target nucleic acid. The amount of QIPC DNA used was 360,000 copies/reaction. QIPC primer and probe concentration was at 36 nM and 150 nM, respectively.

Duplicates for each West Nile Virus RNA concentration and heparin concentration were tested. Table 10 shows that at 0.1× heparin (6 USP units/ml) the QIPC Ct value became “undetermined” compared to the Ct values obtained with the ABI IPC kit which still showed values between 30.98 to 31.79 [less than or just about 3 Ct value difference to the “no inhibitor” Ct values of 28.53 (high target copy) to 28.71 (low target copy)]. In a clinical operations setting, the ABI IPC values would have been reported as “Passed” and the target sequence (at 1,000 copies/mL) would have been reported as “Not detected.” Consequently, the ABI IPC kit showed a false negative for samples of WNV at low copy number when the sample contained heparin at 0.1×. In contrast, the QIPC would have been reported as “Failed” for samples containing 0.1× heparin, and would not have resulted in the sample being identified as a false negative.

In the ABI IPC assay at 100,000 copies/mL of West Nile Virus RNA, the Ct value for the target sequence was 47.10, which is a borderline Ct value that could come out “undetermined” if more replicates were run. The QIPC system, on the other hand, unequivocally showed that Ct values for both QIPC DNA and target sequence were “undetermined”, indicating that there was inhibition of the PCR amplification at both high and low target copy numbers.

TABLE 10
Effect of Heparin on Ct Value of IPG and Target Nucleic Acid

| | 100,000 copies/mL | | | | 1,000 copies/mL | | | |
|---|---|---|---|---|---|---|---|---|
| Heparin | West Nile Virus RNA | | | | West Nile Virus RNA | | | |
| conc. | IPC | WNV | QIPC | WNV | IPC | WNV | QIPC | WNV |
| 1x (60 USP | UD | UD | UD | UD | UD | UD | UD | UD |
| units/ml) | | | | | | | | |
| 0.5x | UD | UD | UD | UD | UD | UD | UD | UD |
| 0.2x | 47.60 | UD | UD | UD | 47.81 | UD | UD | UD |
| 0.1x | 30.98 | 47.10 | UD | UD | 31.79 | UD | UD | UD |
| 0.01x | 27.55 | 28.41 | 29.72 | 28.46 | 27.78 | 35.96 | 29.62 | 35.69 |
| 0.001x | 27.18 | 27.73 | 27.51 | 27.98 | 27.69 | 34.82 | 27.50 | 34.59 |
| 0.0001x | 28.10 | 28.07 | 30.19 | 28.88 | 28.42 | 35.00 | 29.99 | 35.50 |
| 0.00001x | 28.12 | 28.19 | 31.11 | 29.47 | 28.71 | 34.78 | 30.84 | 36.25 |
| 0 | 28.53 | 28.52 | 31.44 | 29.68 | 28.71 | 35.18 | 31.49 | 36.73 |

IPC = ABI IPC Kit; UD = Undetermined (no amplification)

### Example 9

### QIPC2 Primers

This Example describes the use of the Quantitative Internal Positive Control 2 (QIPC2) primer and probe mix for use in TaqMan® assays. The QIPC2 primer/probe mix still uses the same QIPC DNA as template as described in the previous examples. However, primers QIPC2_L and QIPC_R and probe QIPC2_VN (VIC™ dye and NFQ quencher) were used. During real-time PCR amplification, the QIPC2 primers hybridize within the QIPC DNA to produce a 60-bp amplicon as opposed to a 150-bp amplicon produced with the original QIPC primers.

QIPC2 primers (45 nM each) and probe (150 nM) were used in a PCR reaction with QIPC template DNA as described above. Table 11 shows the results of amplification in 10 replicate samples. The Ct values obtained with the QIPC2 primers were similar to those obtained from the QIPC primers described above.

TABLE 11
Optimization of QIPC DNA Concentration.

| Replicate | Ct |
|---|---|
| 1 | 23.740 |
| 2 | 23.980 |
| 3 | 24.150 |
| 4 | 24.360 |
| 5 | 24.580 |
| 6 | 24.740 |
| 7 | 24.640 |
| 8 | 24.220 |
| 9 | 24.050 |
| 10 | 23.730 |
| Avg Ct | 24.219 |
| St Dev Ct | 0.359 |
| % Cv | 1.481 |

Unless otherwise defined, all technical and scientific terms used herein have the same meaning as commonly understood by one of ordinary skill in the art to which this invention belongs,

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims

What is claimed is:

1. A method of quantifying the amount of a target nucleic acid present in a mammalian sample by using quantitative amplification of the target nucleic acid if such sample may contain a non-specific inhibitor of nucleic acid amplification comprising:
(a) contacting a control nucleic acid with the sample to be assayed for the presence of a target nucleic acid, wherein the control nucleic acid comprises a nucleotide sequence from a plant gene;
(b) amplifying the control nucleic acid with a control primer pair which is complementary to the control nucleic acid;
(c) amplifying the target nucleic acid with one or more target primer pairs which are complementary to the one or more target nucleic acids; and
(d) measuring the amount of an amplification product produced by the control primer pair and by the target primer pair,
wherein a reduction in the amount of the amplification product produced by the control primer pair relative to the amount of amplification product produced under similar conditions by the control primer pair without a nucleic acid amplification inhibitor present indicates the effect of any non-specific inhibitor in the mammalian sample; and wherein the amount of amplification product measured for the target primer pair is adjusted to compensate for reduction in control nucleic acid amplified as a result of any non-specific inhibitor in the mammalian sample, whereby the amount of a target nucleic acid present in the mammalian sample is quantified.

2. The method of claim 1, wherein the plant gene encodes ribulose-1,5-bisphosphate carboxylase oxygenase large subunit N-methyltransferase.

3. The method of claim 1, wherein the control nucleic acid comprises from about 40 to about 1480 contiguous nucleotides of SEQ ID NO: 1

4. The method of claim 1, wherein the control nucleic acid comprises a nucleic acid according to SEQ ID NO: 2.

5. The method of claim 4, wherein one or both of the primers of the control primer pair comprises a sequence selected from the group consisting of SEQ ID NO: 5, SEQ ID NO: 6, SEQ ID NO: 8, and SEQ ID NO: 9.

6. The method of claim 1, wherein the measuring is accomplished using labeled oligonucleotide probes complementary to the amplification products produced by the control primer pair and the target primer pair.

7. The method of claim 6, wherein the oligonucleotide probe complementary to the amplification products produced by the control primer pair comprises a sequence selected from the group consisting of SEQ ID NO: 7, SEQ ID NO:10, and SEQ ID NO:11.

8. The method of claim 1, wherein from about 300,000 to about 400,000 copies of the control nucleic acid are contacted with the sample.

9. A method of detecting if a non-specific inhibitor is present in a sample comprising:
(a) contacting a control nucleic acid with a sample to be assayed for the presence of one or more target nucleic acids, wherein the control nucleic acid comprises from about 40 to about 1480 contiguous nucleotides of SEQ ID NO: 1;
(b) amplifying the control nucleic acid with a control primer pair which is complementary to the control nucleic acid;
(c) detecting an amplification product produced by the control primer pair, wherein a reduction in the amount of the amplification product produced by the control primer pair relative to the amount of amplification product produced under similar conditions by the control primer pair without a nucleic acid amplification inhibitor present indicates if a non-specific inhibitor is present in the sample.

10. The method of claim 1, further comprising the step of amplifying one or more target nucleic acids, if present in the sample, with one or more target primer pairs which are complementary to the one or more target nucleic acids.

11. The method of claim 1, wherein the control nucleic acid comprises a nucleic acid according to SEQ ID NO: 2.

12. The method of claim 11, wherein one or both of the primers of the control primer pair comprises a sequence selected from the group consisting of SEQ ID NO: 5, SEQ ID NO: 6, SEQ ID NO: 8, and SEQ ID NO: 9.

13. The method of claim 9, wherein the detecting is accomplished using a labeled oligonucleotide probe complementary to the amplification product.

14. The method of claim 13, wherein the oligonucleotide probe comprises a sequence selected from the group consisting of SEQ ID NO: 7, SEQ ID NO: 10, and SEQ ID NO: 11.

15. The method of claim 9, wherein from about 300,000 to about 400,000 copies of the control nucleic acid are contacted with the sample.

16. A composition comprising:
(a) an isolated control nucleic acid; wherein the control nucleic acid comprises from about 40 to about 1480 contiguous nucleotides of SEQ ID NO: 1 or its complement;
(b) a control primer pair which is complementary to the control nucleic acid; and
(c) a labeled oligonucleotide probe complementary to the control nucleic acid.

17. The composition of claim 16, wherein the control nucleic acid comprises a nucleic acid according to SEQ ID NO: 2.

18. The composition of claim 17, wherein one or both of the primers of the control primer pair comprises a sequence selected from the group consisting of SEQ ID NO: 5, SEQ ID NO: 6, SEQ ID NO: 8, and SEQ ID NO: 9.

19. The composition of claim 16, wherein the oligonucleotide probe comprises a sequence selected from the group consisting of: SEQ ID NO: 7, SEQ ID NO: O, and SEQ ID NO: 1.

20. The composition of claim 16, wherein the composition comprises from about 300,000 to about 400,000 copies of the control nucleic acid.

21. A method comprising:
(a) contacting the composition of claim 16 with a sample to be assayed for the presence of one or more target nucleic acids in an amplification mixture under conditions wherein the control primers specifically hybridize and amplification products of the control nucleic acid are produced;
(b) detecting the amplification products produced by the control primer pair, wherein the presence of the amplification product indicates that a non-specific PCR inhibitor is absent from the amplification mixture.

22. The method of claim 21, wherein the amplification mixture comprises a sample to be tested for a target nucleic acid and a primer pair complementary to the target nucleic acid.

23. The method of claim 21, wherein the amplification mixture comprises deoxynucleotide triphosphates and a DNA polymerase.

24. The method of claim 21, wherein the amplification is performed using real-time PCR.

25. The method of claim 21, wherein the control nucleic acid comprises a nucleic acid according to SEQ ID NO: 2.

26. The method of claim 25, wherein one or both of the primers of the control primer pair comprises a sequence selected from the group consisting of SEQ ID NO: 5, SEQ ID NO: 6, SEQ ID NO: 8, and SEQ ID NO: 9.

27. The method of claim 21, wherein the detecting is accomplished using a labeled oligonucleotide probe complementary to the amplification product.

28. The method of claim 27, wherein the oligonucleotide probe comprises a sequence selected from the group consisting of: SEQ ID NO: 7, SEQ ID NO:10, and SEQ ID NO:11.

29. The method of claim 21, wherein from about 300,000 to about 400,000 copies of the control nucleic acid are contacted with the amplification mixture.


SEQUENCE LISTING (11 sequences)

SEQ ID NO: 1 - DNA, 1480 bp, Arabidopsis thaliana
atgtcagcctccgtcgccgtcgtgtcgggttttctccgtatcccgtcaattcaaaagtctcaaaacccttcctttctcttttctcgacccaagaaatctttggtgagacccatttcagcttcaagctcagagttgccggaaaatgttcggaatttctggaaatggctcagagaccaaggagtcgtatccgggaagtctgtggcagaacctgcggtggtaccggaaggtttgggacttgtagccaggcgtgatattggaagaaacgaggtcgtattggagattcccaagcgattgtggataaacccagagacagtgactgcttccaagattggacctttatgcggcggattaaagccgtgggtttcagtagctctgtttttgatcagagagaagtatgaagaagagtcttcatggagagtttatcttgatatgcttcctcaatctactgattctactgtcttctggtcagaagaggagcttgctgagcttaaagggactcaactgttgagcaccacattgggtgtgaaagagtatgtggagaatgaattcttgaaactggaacaagagatattactgcctaacaaagatctcttctcatcccgcataacacttgatgacttcatatgggcgtttgggatcctcaagtcgagggctttttctcgtctccgtggccaaaaccttgtcttgatccctcttgcagacttgataaaccataaccccgcgataaagacagaagattatgcatacgagatcaaaggagccggccttttctctagagatctcttattttccttgaagtcacctgtttatgttaaagcaggtgagcaggtatacattcagtacgatctgaacaaaagcaatgcagaacttgctctcgactatggttttgtggaatcaaaccctaaacggaactcatatactttaacaatagagataccagaatcagacccattctttggggataagttggatattgctgagagtaacaagatgggtgagaccggatactttgacatagtagacggccagactcttcccgctggtatgcttcagtaccttcggcttgtggctcttggcggtccagatgctttcttattagaatctatcttcaataacaccatatggggtcatcttgaattgcctgtaagtcgtacaaacgaggaactcatatgccgtgttgtcagagatgcctgcaaatctgctctgtctggttttgatacgaccattgaagaggatgagaagcttctggacaaaggaaagcttgagcctaggttggaaatggctctcaagataaggattggtgagaagagagtgcttcagcaaatcgaccaaatcttcaaggatagagagcttgaacttgacattttagagtattaccaagagagaaggctcaaagatcttgggttggttggcgaacaaggggatattatcttctgggaaaccaagtgagaattagcttagcttgtatagcttatcctgc

SEQ ID NO: 2 - DNA, 577 bp, Arabidopsis thaliana
taaccccgcgataaagacagaagattatgcatacgagatcaaaggagccggccttttctctagagatctcttattttccttgaagtcacctgtttatgttaaagcaggtgagcaggtatacattcagtacgatctgaacaaaagcaatgcagaacttgctctcgactatggttttgtggaatcaaaccctaaacggaactcatatactttaacaatagagataccagaatcagacccattctttggggataagttggatattgctgagagtaacaagatgggtgagaccggatactttgacatagtagacggccagactcttcccgctggtatgcttcagtaccttcggcttgtggctcttggcggtccagatgctttcttattagaatctatcttcaataacaccatatggggtcatcttgaattgcctgtaagtcgtacaaacgaggaactcatatgccgtgttgtcagagatgcctgcaaatctgctctgtctggttttgatacgaccattgaagaggatgagaagcttctggacaaaggaaagcttgagcctaggttggaaatggctctcaag

SEQ ID NO: 3 - DNA, 20 bp, Artificial Sequence
  Description of Artificial Sequence Synthetic primer
taaccccgcgataaagacag

SEQ ID NO: 4 - DNA, 20 bp, Artificial Sequence
  Description of Artificial Sequence Synthetic primer
cttgagagccatttccaacc

SEQ ID NO: 5 - DNA, 19 bp, Artificial Sequence
  Description of Artificial Sequence Synthetic primer
cttcagtaccttcggcttg

SEQ ID NO: 6 - DNA, 20 bp, Artificial Sequence
  Description of Artificial Sequence Synthetic primer
ttgcaggcatctctgacaac

SEQ ID NO: 7 - DNA, 21 bp, Artificial Sequence
  Description of Artificial Sequence Synthetic probe
tggctcttggcggtccagatg

SEQ ID NO: 8 - DNA, 18 bp, Artificial Sequence
  Description of Artificial Sequence Synthetic primer
cagactcttcccgctggt

SEQ ID NO: 9 - DNA, 18 bp, Artificial Sequence
  Description of Artificial Sequence Synthetic primer
atctggaccgccaagagc

SEQ ID NO: 10 - DNA, 23 bp, Artificial Sequence
  Description of Artificial Sequence Synthetic probe
tgcttcagtaccttcggcttgtg

SEQ ID NO: 11 - DNA, 23 bp, Artificial Sequence
  Description of Artificial Sequence Synthetic probe
tgcttcagtaccttcggcttgtg